(19) United States
(12) Patent Application Publication
(10) Pub. No.: US 2016/0287796 A1
(43) Pub. Date: Oct. 6, 2016

(54) CARTRIDGE SYSTEM TO WHICH A SYRINGE BODY CAN BE ATTACHED

(71) Applicant: XEND MEDICAL SYSTEMS, LLC, Salt Lake City, UT (US)

(72) Inventors: Randall J. Olson, Salt Lake City, UT (US); Owen D. Brimhall, South Jordan, UT (US); Jeremy Hammer, Salt Lake City, UT (US); Andrew G. Raguskus, Salt Lake City, UT (US)

(21) Appl. No.: 14/928,701

(22) Filed: Oct. 30, 2015

Related U.S. Application Data
(60) Provisional application No. 62/142,386, filed on Apr. 2, 2015.

(51) Int. Cl. A61M 5/31; A61M 5/32

(52) U.S. Cl. CPC A61M 5/31; A61M 5/32; A61M 2005/3117; A61M 2005/3118; A61M 2005/312

(57) ABSTRACT

A needle hypodermic system and method for reducing the incidence of infections due to administering fluids by injection or for reducing the risk of surface contamination of an aspirated sample of blood or other bodily tissue in humans or animals is provided. The hypodermic needle system may include a needle with a plug or film disposed over or within its hollow bore to substantially prevent bacteria, fungi and/or other organisms/contaminants, which may be present on the exterior surface to be penetrated, from adhering to the inner surfaces of the needle's channel or become trapped in the needle bore. A second stylet or needle may be deployed to pierce or push aside the plug.

### BACKGROUND OF THE INVENTION

### State of the Art

The present invention relates to a hypodermic needle system and a method to reduce the incidence of infection or contamination caused when the needle is inserted into a human or animal. In some embodiments, the present invention relates to a hypodermic needle with a second needle or stylet disposed in the hollow channel of the first needle, along with a plug or membrane blocking the opening of the first needle to protect the primary needle channel from becoming contaminated prior to insertion of the second needle tip into a blood vessel or other location in the body to either inject fluids or withdraw fluids from the body.

There is a variety of situations in which a needle is inserted into a human or animal. When most people think of the use of a hypodermic needle, they think of administering a compound to achieve a therapeutic effect in humans or animals. Injecting a drug into its site of action increases the potential effectiveness of the drug. However, each injection carries a small risk of infection to the patient. As the needle penetrates the skin, the needle can carry bacteria and other pathogens into the patient. Infection due to injections can often be decreased by using proper aseptic technique, such as by swabbing the region to be injected with antibacterial solution. However, few technicians wait the required 90 seconds for the solution to take full effect and elimination of all skin bacteria is difficult and rarely occurs. Additionally, there are a variety of procedures in which a needle must be inserted into the body through a tissue which cannot be readily sterilized, such as injections which are made into the eye or mucus membranes.

However, because other methods of administering the drug may be less effective or not effective at all, it may be required to inject the drug anyway to effectively treat a particular disease or condition. This is often the case, for example, when treating eye diseases or conditions because the blood-ocular barrier keeps most drugs out of the eye, and because tolerable sterilizing solutions are not very effective on the conjunctival surface.

Under normal circumstances, the blood-ocular barrier protects a human or animal by providing natural resistance against organisms invading the vitreous humor, the clear gel that fills the space between the lens and the retina of the eyeball. The immune response of a human or animal to an organism that is introduced into the vitreous humor is more limited than if the organism was present in other areas of the body. Thus, a medical procedure that disrupts the integrity of the globe of the eye, such as intravitreal injections to treat a disease or condition of the eye, can lead to infection and inflammation of the eye, i.e. endophthalmitis.

It has been found that when the needle passes through the exterior membranes surrounding the eyeball (the conjunctiva), bacteria, which are present normally, can be introduced into the interior hollow channel of the needle and ultimately deposited in the vitreous humor when a substance is injected.

Moreover, because the conjunctiva cannot be readily sterilized prior to intravitreal injections the risk of infection is not as minimal as is desired with such injections. Some common complications of endophthalmitis are decreased vision and/or permanent vision loss. Some patients may even require enucleation (removal of the entire eye) to eradicate a blind and painful eye.

Intravitreal injection of various drugs has become a mainstay of treatment in ophthalmology. It is currently estimated that approximately 1000 to 3000 infections due to intravitreal injection occur each year with approximately half of those infections resulting in legal blindness. The number of injections given each year is increasing as the understanding of how to treat certain eye diseases or conditions increases, and/or new drugs for treating such diseases or conditions become available. For example intravitreal injections may be given to treat viral retinitis, age-related macular degeneration, cystoid macular edema, diabetic retinopathy, uveitis, vascular occlusions, and even endophthalmitis. For the most common condition, wet macular degeneration, injections may have to be given monthly for the rest of the patient's life so that the cumulative risk of infection becomes substantial.

Even during injections involving areas other than the eye, infection and contamination are concerns. Needle bores have been shown to collect bacterial contamination during the puncturing process. In a person with a compromised immune system, the bacteria or other microbes can be carried into the body and cause infections.

When administering a pharmaceutical compound or other therapeutic agent to a human or animal by injection, or when inserting a needle to take a blood sample, the tissue surface that is to be penetrated by the needle is typically sterilized with a chemical, such as alcohol, iodine, etc. In some areas of the body, such as a mucous membrane like the conjunctiva that covers the eye, some chemicals cannot be or are difficult to use. In other areas, such as the skin, it is common for the person inserting the needle not to wait a sufficient amount of time for the antiseptic to kill all of the bacteria and many bacteria in sweat glands, hair follicles and the deeper epidermis usually survive even if the antiseptic is used appropriately. Thus, it is fairly easy for the needle to become contaminated.

The failure to properly sterilize the skin also raises the risk of false positives when taking blood samples to determine if there is a blood borne infection. This is at greatest risk when the bacteria enter the bore of the needle because the layers of skin typically mechanically clean the exterior of the needle as it advances through the tissue. The interior, however, may simply become filled with bacteria and other microbes which can either be injected into the patient when administering medicine, or drawn back into the syringe when taking a blood sample. This is a very common clinical quandary

The ability of contaminating microbes to be drawn back into the syringe is another major problem in medicine. Studies suggest that, depending on the medical facility, 0.6% to 6 percent of blood tests are contaminated by bacterial and other microbes which are not actually in the blood. (Hall and Lyman, Updated Review of Blood Culture Contamination, Clinical Microbology Reviews, October 2006, pages 788-802.) This is significant because bacteremia (the presence of bacteria in the blood), besides potentially causing wide spread bacterial infection throughout the body, can cause the immune system to release chemicals which can lead to widespread inflammation. The inflammation may result in blood clotting and organ damage. In some cases, the patient suffers a dramatic drop in blood pressure (septic shock) and organs shut down causing death. Each year sepsis kills more than 258,000 Americans and it ranked by the Center for Disease Control as the ninth leading cause of disease related deaths. www.cdc.goc/spsis/basic/qa.html (Oct. 13, 2015).

Because of the potentially devastating effects of sepsis, hospitals take bacteremia and the like very seriously. Patients showing symptoms consistent with bacteria in the blood have blood tests done to ensure that they are not suffering from a blood infection. If the blood tests confirm bacteria, etc., in the blood, the patient will be admitted or held as an in-patient for several extra days to address the infection.

While hypodermic needles are a necessary tool in drawing blood for such tests, they also cause a significant financial loss for hospitals. In the process of puncturing the skin, the bore of the needle will often trap bacteria from the skin inside the bore of the needle. When blood is then drawn through the needle, the bacteria mixes with the blood supply from the patient. When the blood is tested, a positive result for bacterial infection is received even though the patient does not actually have sepsis. Because hospitals often receive flat fee reimbursements and the rate of false positives is so high, having patients who are not suffering from sepsis remain in the hospital for additional days can cost hospitals billions of dollars per year. The patients who must remain in the hospital due to these false-positive blood tests lose workdays, and are exposed to further infections in a hospital environment.

A number of efforts have been made to help differentiate false positives for bacteremia from actual infections. These include attempts to differentiate risk based on organism detected, the number of blood cultures testing positive, and time to growth of the bacteria. (Hall and Lyman, Updated Review of Blood Culture Contamination, Clinical Microbiology Reviews, October 2006, pages 788-802.) Each of these, however, is only predictive and runs the risk that a person actually suffering from bacteremia is inaccurately determined to be a false positive.

Thus, there is a need for a hypodermic needle system and method that substantially minimizes the transmission of bacteria and the like when a needle is advanced through the skin or other external tissues of a person or animal. It is desirable that such a hypodermic needle system is relatively easy to use. It is also desirable to provide such a hypodermic needle system which is inexpensive and easy to manufacture.

### SUMMARY OF THE INVENTION

It is an object of the present invention to provide a hypodermic needle system and method of use to reduce the incidence of iatrogenic infection and/or to reduce the rate of false positives in blood tests.

The present disclosure includes multiple different devices, systems, methods and applications which can reduce the incidence of iatrogenic infection and/or reduce the rate of false positives in blood tests and are thus applications of a common inventive concept. It should be appreciated that various devices, systems, methods and applications will have some benefits and may lack other benefits which are present in different devices, systems, methods and applications. Therefore, the teachings of the present disclosure and any actual or intended benefit of any embodiments should not be read into the claims unless expressly stated therein.

According to one aspect of the present disclosure, a hypodermic needle system may include a first needle and a plug disposed within the hollow bore of the first needle and configured to substantially obstruct the channel at the penetrating end of the needle. The hypodermic needle system may further include a stylet adapted to fit inside the hollow bore or lumen of the first needle. The stylet may be, for example, a second needle which may be slidable independent of the first needle.

According to another aspect of the present disclosure, the plug of the hypodermic needle system may substantially prevent bacteria, fungi, or other organisms/contaminates from entering the hollow bore or lumen of the needle.

According to another aspect of the present invention, the plug of the hypodermic needle system may be generally convex outside of the bore so as to provide a rounded surface which limits the ability of bacteria to attach to or reside in crevices or indentations in the plug and which promotes mechanical wiping of the exterior of the plug as the needle tip is passed through the layers of the skin.

In one embodiment of the present disclosure, the plug is dissolvable. For example, the dissolvable plug may, in some embodiments, be formed of a microbial resistant, biodegradeable, biocompatible material such as PLGA (polylactic-co-glycolic acid).

In one embodiment the biodegradable or bioabsorbable plug material may be formed with a therapeutic agent.

In one embodiment the biodegradable plug material may be formed with a therapeutic agent with a specified time release dissolution rate.

According to still another aspect of the present disclosure, the plug of the hypodermic needle system may be disposed in alignment with the beveled end of the hypodermic needle.

According to yet another aspect of the present disclosure, the plug of the hypodermic needle system may be removable. In some embodiments, the plug or similar insert may be pushed out of the lumen of the first needle or penetrated centrally by the tip of the stylet, e.g. a wire or a second slidable needle.

According to yet another aspect the plug may be pushed out of the lumen but be retained by the tip of the stylet, e.g. with a wire or second slidable member to allow retention upon removal of the needle assembly.

According to yet another aspect of the present disclosure, the plug may be physically attached to the tip of the stylet so that the plug may be physically removed without pushing out the plug but by pulling out the plug.

According to yet another aspect of the present disclosure, the plug may be chemically designed to dissolve in a short amount of time within the bore of the needle once it has been in contact with the desired tissue so that no pushing or pulling of the plug is needed for it to dissolve. It simply dissolves in its place in the bore due to its chemical properties thereby eliminating the need for a stylet to remove the plug.

According to another aspect of the present disclosure, the hypodermic needle system may include a needle with a bent tip. The tip of the needle may be bent at the bevel so that penetration can be made with a linear puncture.

In accordance with another aspect of the present disclosure, a hypodermic needle system may be provided comprising a needle having a beveled end and a plug having a beveled face, wherein the beveled end of the needle and the beveled face of the insert are positioned relative to each other so as to form a substantially single, planar surface.

According to still another aspect of the present disclosure, a hypodermic needle system may include a film covering a surface of the penetrating end of the hypodermic needle system. The film may reduce adherence of bacteria, fungi, or other organisms/contaminates to the surfaces of the hypodermic needle system. The film may be supported by a plug portion disposed within the lumen of the first needle to prevent the film from breaking as it is advanced through the tissue.

In yet another aspect of the present disclosure, the stylet may form the penetrating tip to a blunt needle, with a seal disposed between the stylet and the outer needle. With the stylet advanced the needle bore opens so that medicine can be injected or blood or other fluid sample can be aspirated. In accordance with one embodiment, the seal is formed from PLGA.

In accordance with one aspect of the present disclosure, the film of the hypodermic needle system may be a thermoplastic such as poly methyl methacrylate (“PMMA”), cyanoacrylate compounds, or any other suitable material to which infectious agents have a reduced ability to adhere as compared to the surface of a needle, or other substance that resists adherence of bacteria, fungi, or other organisms/contaminates to the surfaces of the hypodermic needle system and substantially eliminates any space where microorganisms may be trapped. The film may also be formed from a bio-dissolvable material.

According to another aspect of the disclosure, the hypodermic needle system may comprise an antibiotic, antifungal, or sterilizing compound.

According to another aspect of the disclosure, a hypodermic needle system may include a needle attached to a medicament container, such as a syringe, wherein the needle may comprise a removable insert or stylet disposed within the hollow channel of the needle, and wherein the insert may be removed from the hollow channel of the needle while the needle is attached to the medicament container.

According to another aspect of the disclosure, a hypodermic needle system may contain a double-headed needle hub in which one tube may contain the inner stylet and the other tube may contain a closed system within the double-headed hub.

In accordance with one aspect of the present disclosure, a method of reducing the incidence of infections due to administering fluids by injection to humans or animal may comprise selecting a syringe having a plug within the hollow channel of a needle prior to penetrating an exterior surface of the human's or animal's body so as to substantially reduce the amount of bacteria, fungi and/or other organisms/contaminants that may be transmitted from an external surface to any internal area of the human's or animal's body, then removing the plug after the needle has penetrated the external surface to administer the fluid, by pushing or penetrating the plug out of the hollow channel of the needle with a second slidable needle. In other embodiments, the second needle or stylet may be fixed, and the first needle may be slidable, so that the first needle can be drawn back to expose the tip of the second needle thereby pushing the insert or plug out of the first needle.

According to another aspect of the disclosure, a hypodermic needle system may include a hermetically sealed needle tip, the tip being sealed using a biocompatible and bio-absorbable material such that the seal, or fragments of the seal, can be left in the body.

According to another aspect of the present disclosure, various methods are disclosed to hermetically seal the needle tips with a biocompatible and bio-absorbable material. Also disclosed is the infusion of these biocompatible materials with various bioactive materials such as a drug, medication, or anti-infective agent.

According to still another aspect of the present disclosure, a spacer clip or a slider is disclosed for positioning first needle and a stylet an appropriate distance apart. In this manner, a standard syringe may be fitted with a first, outer needle and a second, inner needle.

In some embodiments, the hypodermic needle system may include a blunt-tipped second stylet, which may improve the process of ejecting the biocompatible plug or insert from the outer needle bore.

These and other aspects of the present invention are realized in a hypodermic needle system and method of use as shown and described in the following figures and related description.

### BRIEF DESCRIPTION OF THE DRAWINGS

Various embodiments of the present invention are shown and described in reference to the numbered drawings wherein:

FIG. 1 shows a cross-sectional side view of a hypodermic needle system in accordance with some aspects of the present disclosure;

FIG. 2 shows a cross-sectional view of a hypodermic needle system with an alternate plug disposed within the hollow channel of a needle;

FIG. 3 shows a cross-sectional side view of the hypodermic needle system of FIG. 2 with the plug ejected;

FIG. 4 shows a cross-sectional view of a hypodermic needle system with a plug disposed in the hollow channel of a needle, and a depth stop disposed on the outer surface of the first needle;

FIGS. 5 and 6 show a first needle attached to a syringe-sleeve device formed in accordance with the present disclosure, adapted to receive a standard syringe with a stylet and to keep the first needle and the stylet at an appropriate distance apart until a user decides to eject the plug;

FIG. 7 shows a view of a spacer clip adapted to hold a first outer needle at a desired spacing relative to a second inner needle;

FIG. 8 shows a close-up view of the spacer clip of FIG. 7 as the syringe is being mounted in the spacer clip;

FIGS. 9 and 10 illustrate the spacer clip of FIG. 7 affixed to a standard syringe to hold a first outer needle at a desired spacing from a second inner needle, while both are affixed to a standard syringe;

FIG. 11 shows a top view of the syringe and outer needle of FIGS. 8 through 10 with the spacer clip having been removed.

FIGS. 12 and 13 show side cross-sectional views of an alternate configuration of a needle having a movable stylet and plug with the plug in retracted positions and extended positions, respectively;

FIG. 14 shows a side cross-section view of an alternate plug;

FIGS. 15 and 16 shows an alternative configuration wherein the plug remains attached to the needle during use;

FIG. 17 shows a fragmented, side cross-sectional view of an alternate configuration of a syringe system in accordance with one aspect of the invention in a closed position, wherein the stylet forms the plug.

FIG. 18 shows a fragmented, side cross-sectional view of the embodiment of FIG. 17 in an open position;

FIG. 19 shows a top view of an alternate embodiment of a syringe system made in accordance with another aspect of the invention; and

FIG. 20 shows the syringe system of FIG. 19 with the stylet advanced to puncture the septum and a sealing membrane.

It will be appreciated that the drawings are illustrative and not limiting of the scope of the invention which is defined by the appended claims. The various elements of the invention accomplish various aspects and objects of the invention. It is appreciated that not every element of the invention can be clearly displayed in a single drawing, and as such not every drawing shows each element of the invention.

### DETAILED DESCRIPTION

The drawings will now be discussed in reference to the numerals provided therein so as to enable one skilled in the art to practice the present invention. The drawings and descriptions are exemplary of various aspects of the invention and are not intended to narrow the scope of the appended claims. It will be appreciated that the various aspects of the hypodermic needle systems discussed herein may be the same. Different reference numerals may be used to describe similar structures in the various hypodermic needle systems for clarity purposes only.

Various aspects of the invention and accompanying drawings will now be discussed in reference to the numerals provided therein so as to enable one skilled in the art to practice the present invention. The skilled artisan will understand, however, that the methods described below can be practiced without employing these specific details, or that they can be used for purposes other than those described herein. Indeed, they can be modified and can be used in conjunction with products and techniques known to those of skill in the art in light of the present disclosure. Furthermore, it will be appreciated that the drawings may some aspects of the invention in isolation and the elements in one figure may be used in conjunction with elements shown in other figures.

Reference in the specification to “one configuration,” “one embodiment” “one aspect” or “a configuration,” “an embodiment” or “an aspect” means that a particular feature, structure, or characteristic described in connection with the configuration may be included in at least one configuration and not that any particular configuration is required to have a particular feature, structure or characteristic described herein unless set forth in the claim. The appearances of the phrase “in one configuration” or similar phrases in various places in the specification are not necessarily all referring to the same configuration, and may not necessarily limit the inclusion of a particular element of the invention to a single configuration, rather the element may be included in other or all configurations discussed herein. Thus it will be appreciated that the claims are not intended to be limited by the representative configurations shown herein. Rather, the various representative configurations are simply provided to help one of ordinary skill in the art to practice the inventive concepts claimed herein.

Furthermore, the described features, structures, or characteristics of embodiments of the present disclosure may be combined in any suitable manner in one or more embodiments. In the following description, numerous specific details may be provided, such as examples of products or manufacturing techniques that may be used, to provide a thorough understanding of embodiments of the invention. One skilled in the relevant art will recognize, however, that embodiments discussed in the disclosure may be practiced without one or more of the specific details, or with other methods, components, materials, and so forth. In other instances, well-known structures, materials, or operations may not be shown or described in detail to avoid obscuring aspects of the invention.

Before the present invention is disclosed and described in detail, it should be understood that the present invention is not limited to any particular structures, process steps, or materials discussed or disclosed herein. More specifically, the invention is defined by the terms set forth in the claims. It should also be understood that terminology contained herein is used for the purpose of describing particular aspects of the invention only and is not intended to limit the invention to the aspects or embodiments shown unless expressly indicated as such. Likewise, the discussion of any particular aspect of the invention is not to be understood as a requirement that such aspect is required to be present apart from an express inclusion of that aspect in the claims.

It should also be noted that, as used in this specification and the appended claims, singular forms such as “a,” “an,” and “the” may include the plural unless the context clearly dictates otherwise. Thus, for example, reference to “a bracket” may include an embodiment having one or more of such brackets, and reference to “the target plate” may include reference to one or more of such target plates.

As used herein, the term “substantially” refers to the complete or nearly complete extent or degree of an action, characteristic, property, state, structure, item, or result to function as indicated. For example, an object that is “substantially” enclosed would mean that the object is either completely enclosed or nearly completely enclosed. The exact allowable degree of deviation from absolute completeness may in some cases depend on the specific context, such that enclosing the nearly all of the length of a lumen would be substantially enclosed, even if the distal end of the structure enclosing the lumen had a slit or channel formed along a portion thereof. The use of “substantially” is equally applicable when used in a negative connotation to refer to the complete or near complete lack of an action, characteristic, property, state, structure, item, or result. For example, structure which is “substantially free of” a bottom would either completely lack a bottom or so nearly completely lack a bottom that the effect would be effectively the same as if it completely lacked a bottom.

As used herein, the term “about” is used to provide flexibility to a numerical range endpoint by providing that a given value may be “a little above” or “a little below” the endpoint while still accomplishing the function associated with the range.

As used herein, a plurality of items, structural elements, compositional elements, and/or materials may be presented in a common list for convenience. However, these lists should be construed as though each member of the list is individually identified as a separate and unique member.

Concentrations, amounts, proportions and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually. This same principle applies to ranges reciting only one numerical value as a minimum or a maximum. Furthermore, such an interpretation should apply regardless of the breadth of the range or the characteristics being described.

The invention and accompanying drawings will now be discussed in reference to the numerals provided therein so as to enable one skilled in the art to practice the present invention. The drawings and descriptions are intended to be exemplary of various aspects of the invention and are not intended to narrow the scope of the appended claims. Furthermore, it will be appreciated that the drawings may show aspects of the invention in isolation and the elements in one figure may be used in conjunction with elements shown in other figures.

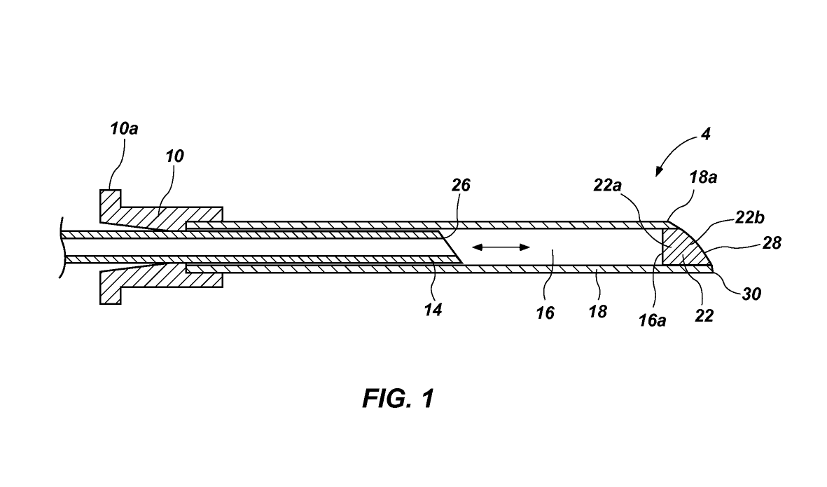

Turning to FIG. 1, there is shown a fragmented, cross-sectional view of a hypodermic needle system, generally indicated at 4, according to principles of the present invention. The hypodermic needle system 4 may include a first, outer needle 18 which is configured to be advanced into a person or animal to either inject a fluid into the person or animal or to withdraw fluid from the person or animal. The first, outer needle 18 may include a base 10 configured for attachment to another structure such as a Luer lock.

The hypodermic needle system 4 may also include an insert or stylet which is moveable within the first needle 18. The insert or stylet 14 may be, for example, a second, inner needle which is hollow, or a structure such as a needle, or other stylet, such as a blunt wire, lance, or probe, used to clear a blockage from first, outer needle 8, as will be discussed below. For portions of the discussion below, the insert or stylet will be a hollow needle and the number 14 will be used therewith.

The insert or second needle 14 may be disposed within the hollow bore or lumen 16 of the first needle 18 so that it is slidable therein. A plug 22 may be disposed in the hollow bore 16 at a first, distal end 16a thereof so as to substantially prevent bacteria, fungi, or other organisms/contaminates from entering the bore and attaching to the inner sidewalls of the needle 18 as the first needle 18 is advanced through the skin. The plug 22 may be comprised of biodegradable plastic, starch, PLGA, or any other microbial resistant, biodegradable, biocompatible material. For reasons which are discussed below, PLGA is a particularly beneficial material from which to make the plug.

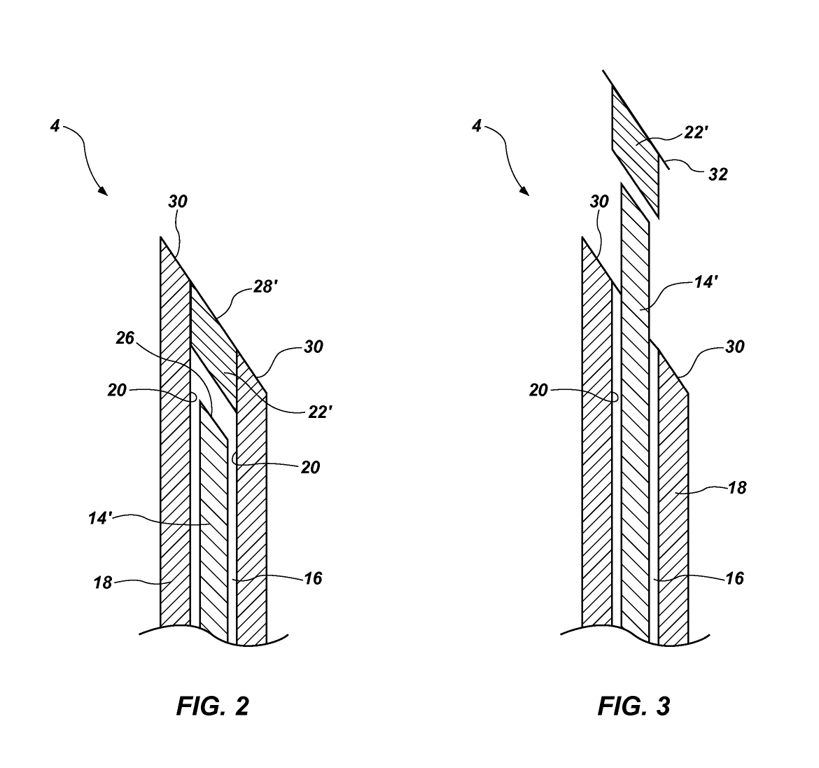

The plug 22 may be inserted into the bore 16 of the first needle 18 or may be formed therein by processes such as by dipping the tip of the first needle 18 in a biocompatible, bio-dissolvable material such as PLGA. The PLGA material can seal the needle bore 16 directly for small bore needles. For larger needles, a plug may be preformed and inserted into the first end 16a of the bore 16 of the first needle 18. As is shown below in FIG. 2, the plug may simply close off the bore 16 of the needle or, as shown in FIG. 1, the plug may cover part of or all of the face of the distal tip of the needle.

A plug 22 made from PLGA or similar materials could be formed in a number of ways. During dipping, the PLGA or other biocompatible material can be dissolved into an appropriate solvent, such as acetone. Various dilutions can be made by varying the ratio of PLGA or other biocompatible material to the solvent. Various viscosities can be made and optimized for the needle diameter and area to be covered and the thickness of the coating desired. Multiple dips can be used to build up material with appropriate drying times between dips. For sealing small gaps a thin, less viscous mix may be adequate.

During injection molding, a syringe or other applicator may be filled with the plug 22 solution. The solution may then be directly injected into the external needle 18 and allowed to cure to form a rigid or semi-rigid plug.

During combination dipping/injection molding, the syringe or other applicator may be filled with viscous PLGA or other biocompatible material such as a glycolide and L-lactide copolymer, which is then directly injected into the external needle 18. Thereafter, a thin, less viscous mix of biocompatible material and solvent may be applied to the distal end 18a of the first, outer needle 18. This may be done by dipping to form a coating, by spray application to form a coating, or by any other means known to one of skill in the art. This combination method has the advantage of forming (by injection molding) a stable plug 22 which does not dislodge during needle penetration into tissue. The additional thin coating may help reduce the incidence of micro-crevasses or irregularities which could harbor bacteria or other contamination. In addition, this method minimizes any blunting of the internal or external needle cutting edges, which might otherwise occur if thicker materials were used.

During micromolding, PLGA or other biocompatible material may be melted and molded or extruded into or onto the bore of the outer needle 18 to form the plug. In this process the solid plastic, PLGA, or other biocompatible material is heated to the glass transition temperature. The PLGA or other biocompatible material can then be pressed or injected into the tip of the internal needle 14 or external needle 18 to form a hermetic seal. A molding tool can be used to extrude and cut the PLGA or other biocompatible material to control the inner and outer geometry of the plug 22. For example the exterior face 28 of the plug 22 can be molded in a convex shape to aid in controlling the ability of bacteria and other microbes from remaining on the plug. The convex form avoids pockets and crevices where microbes could stay and improves the mechanical wiping of the plug as it moves through the tissue. Likewise, a blunt area may be formed on the inner geometry to help in ejection. When formed to desired shape, the plug 22 may then be inserted into the external needle 18 and then heated to form a seal.

The micromolding method may be combined with the dipping or spray methods to fuse or bond the plug.

Further, it will be appreciated that the PLGA or other biocompatible material may be mixed, either while in solution or while heated, with further anti-infective compounds, such as an antifungal, antibacterial, or substances which reduce the risk of bacteria adhesion to the plug 22. The PLGA or other biocompatible material may also be infused with a drug for therapeutic effects.

Use of the hypodermic needle system 4 of the present invention may be particularly beneficial when the needle 18 must be inserted into the body through a tissue which cannot be readily sterilized. The exterior of the needle 18 is mechanically cleaned by the tissue it passes through, thereby limiting the risk of infection. The bore 16 of the needle 18 is protected by the plug 22, which prevents bacteria and other microbes from getting into the bore.

As shown in FIG. 1, the plug 22 may have an internal portion 22a which fills the bore 16 of the first needle 18, and an external portion 22b which extends from the beveled tip 30 at the distal end of the needle. The external portion 22b may have a convex face 28 and extend slightly from the beveled tip 30 of the needle 18. This convex design helps to mechanically wipe clean the plug 22 as it is being advanced through the tissue. By avoiding concave structures and by using a material which bacteria have a hard time clinging to, the risk of bacteria being introduced deep into the patient is substantially reduced.

The insert, stylet or second needle 14 is advanceable within the first needle 18 so as to forcefully engage the plug 22 and either pierce it with a beveled tip 26, or push it out of the lumen 16. Thus, it will be appreciated that the second needle 14 need not have a sharp end as it is the first needle 18 which actually advances through the skin. If the plug 22 is made from a bio-dissolvable material, such as PLGA, the plug can be simply ejected into the patient where it will dissolve over a period of time. The second needle 14 or other insert (e.g. a very small catheter, etc.,) can then be used either to inject medication into the patient, or to withdraw blood samples and the like. Because the plug sealed the first needle 18 during insertion and was mechanically cleaned in the process, the risk that the plug will carry sufficient bacteria to infect the patient or to create false positives within a sample is substantially reduced. (While the second needle 14 may push the plug out, it will be appreciated that the second needle could be used to core the plug—i.e. form a hole therethrough—to allow fluid to pass into our out of the first needle and second needle if desired).

As can also be seen in FIG. 1, the second needle 14 of hypodermic needle system 4 may comprise a beveled face 26 that may be disposed substantially in alignment with the beveled edge 30 of a typical needle 18. The edge 30 of needle 18 is beveled to create a sharp pointed tip letting the needle 18 easily penetrate the tissue surface. Thus, the internal needle 14 can be extended when the primary needle 18 reaches a first desired depth in tissue, the second or internal needle 14 can then access deeper tissues, if necessary, without carrying in any bacteria from the surface of the skin.

Because the second needle 14 is retracted, the second or inner needle 14 does not interfere with penetration, while remaining protected by the plug 22 on the first needle 18. Once penetration has occurred, the second needle 14 can be advanced, thereby pushing the plug 22 out of end 18a of the needle 18, thereby allowing the second, inner needle to either inject fluid or withdraw fluid from the body. If there is concern that the plug will not dissolve quickly enough in the blood stream, the plug 22 could be ejected from the needle 18 and further advancement made by the outer needle or the inner needle without risk of further contamination, as the needles are already past the epidermis.

Depending on the particular desired use of the needle system 4, the insert or stylet can be a second needle 14, a hollow tube, a lance, or other structure. Any of these could be advanced to remove or open the plug 22 from the distal end 16a of the bore 16. In some configurations, the second needle 14 or other stylet could be withdrawn, allowing fluids to be injected or drawn up through the external needle 18. Alternatively, the insert or stylet may be used to inject medicine or withdraw blood, etc., e.g., through the second needle 14.

Now turning to FIG. 2, there is shown a cross-sectional view of a hypodermic needle system, generally indicated at 4, with an alternate plug 22′ disposed within the hollow bore 16 of a needle 18. Plug 22′ is disposed within the hollow bore 16 adjacent the penetrating end of hypodermic needle 18 so as to substantially prevent bacteria, fungi, and/or other contaminants from contacting inner surface 20 of needle 18 which defines the bore 16. Rather than having a convex surface, the plug 22′ has a generally flat, face 28′ which may align with the beveled distal end 30 of the first, outer needle 18 so as to provide a continuous substantially planar surface at the end of the needle. It will be appreciated that other shapes may be used, but it is preferable that the shape not provide crevices or the like where bacteria can be trapped.

It is particularly advantageous if a shape is chosen such that, when insert or internal second needle 14 is deployed, the tip 26′ of internal needle 14 pushes plug 22′ out of the bore 16. (In the alternative, the internal needle may core the plug to allow flow therethrough).

As previously discussed, the plug 22′ may be a biocompatible, biodegradable (dissolvable) material such as PLGA. The plug 22′ can be applied to the internal or external needle by several processes, such as dipping, injection molding, combination, or micromolding, and maybe flattened after formation. Alternatively, the plug may also be attached on the distal end 30 of the first needle 18 by a film or other covering 32 (FIG. 3). While some films are good at resisting the attachment of bacteria, a film alone is problematic because it would not generally stand up to the pressure of being inserted through tissue and would likely fail—allowing bacteria into the bore 16 of the first needle 18. The plug 22′ provides support for the film and prevents the bore 16 from becoming filled with bacteria and the like.

FIG. 2 is also different from FIG. 1 in that the stylet 14′ may be generally solid (though it will be appreciated that a solid stylet could be used in the embodiment shown in FIG. 1 as well). The stylet 14′ (when solid) is there to push the plug out of the bore 16, but not to collect or inject fluid. Rather, that may be done through the first, outer needle 18.

FIG. 3 shows a fragmented, cross-sectional side view the hypodermic needle system 4 with the plug 22′ pushed out of the bore 16. Additionally, the insert or stylet needle 14′ (FIG. 1) itself is deployed beyond the first needle 18. It will be appreciated that any means of deployment may be used for extending the stylet 14′ beyond the first needle 18. For example, deployment means may include an extra depressor button on the body of the syringe (not shown), or by drawing back the first needle 18, etc., so that the stylet 14′ extends beyond the hollow bore 16 of first needle 18 after the first needle 18 has penetrated an exterior tissue surface.

While some prior devices have used a film to cover the end of a needle or other penetrating device, such devices raised various issues. For example, if a film is unsupported, it will likely tear advancing through tissue and allow the entry of potential contaminants While others have suggested providing an insert to support the film and withdraw the film into the syringe with the insert, pulling the film back into the bore of the needle could draw contaminants on the film back into the needle. Thus, while such configurations may be an improvement over prior methods of using needles, the present disclosure further reduces the risk of contamination of the bore.

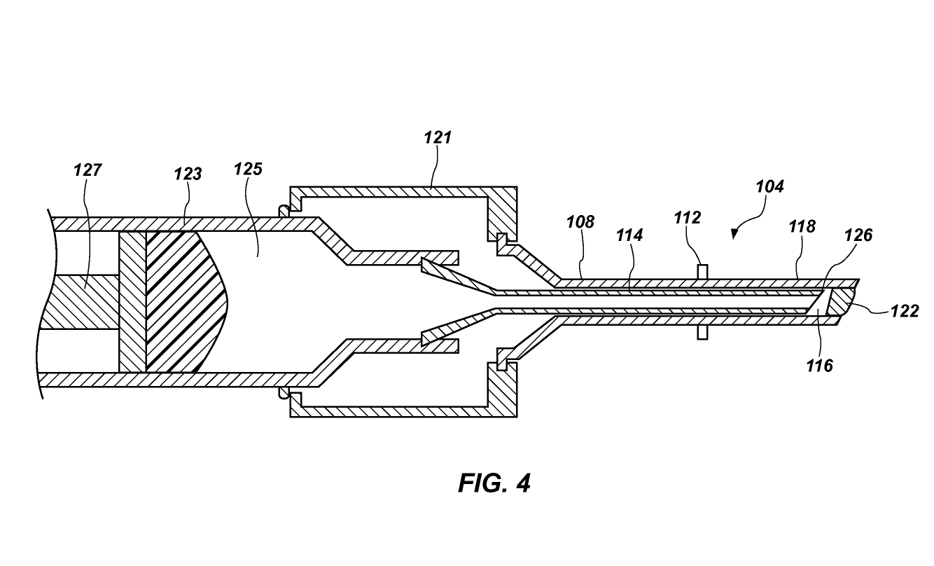

Turning now to FIG. 4, there is shown a side cross-section view of a hypodermic needle system 104 which includes a first, outer needle 108 and an insert in the form of a second, inner needle 114. On the external surface of the first, outer needle 108 may be disposed a depth guide or stopper 112. The depth guide 112 may be a visual indicator, to display to a user how deep the sheathing needle has penetrated—for example, tic marks or a change in color. In other embodiments, the depth guide 112 is a physical stopper, such as a ridge or protrusions, which prevents the first, outer needle 108 from being inserted past a desired depth. Once the desired depth is reached, the second, inner needle 114 is deployed. This may be done, for example, by holding a housing 121 which is attached to the first, outer needle 108 and pushing forward on a syringe body 123 attached to the second, inner needle 114. Advancement of the second, inner needle 114 causes the end 126 of the second, inner needle 114 to press against the plug 122 and move it out of the bore 116 of the first, outer needle 108 or to penetrate the plug centrally. The plug 122, being biodegradable and biocompatible, is left in the tissue but quickly degrades.

In addition, an anti-infective agent (not shown) may be applied to the first, outer needle 118 and/or the plug 122 in order to aid in reducing infections caused by procedures requiring injections. Once the plug is out of the way, the second, inner needle 114 can be used to either inject a fluid 125 in the syringe body 123, or to withdraw blood or other body fluids into the syringe body for testing. Either is accomplished by movement of the plunger 127 in the syringe body.

It will be appreciated that the syringe body 123 and second, inner needle 114 can be removed from the first, outer needle 108 and housing 121. In the alternative, the structures can form a retention mechanism, such as a snap fit between the exterior of the syringe body 123 and the body 121 so that once the second, inner needle 114 is advanced, they stay together.

Many means of deployment of the second, inner needle 114 are possible, such as a trigger, a depressible button, a squeeze lever, a ratcheting tool, a twistable or rotatable extender, etc. It is advantageous if the deployment means permits easy one-handed use, and can be adapted to fit on a standard syringe body 123 or other gripping member.

In some embodiments it will be desirable for the first, outer needle 108 and the second, inner needle 114 to have a snug fit. In other embodiments, however, it may be desirable for the second, inner needle 114 to be sufficiently smaller than the first, outer needle's interior bore that pressing on the plunger 127 causes a small amount of fluid 125 to exit the syringe body 123, travel down the second, inner needle 114 and then back flow through the bore of the first, outer needle toward the housing 121. In such a manner the system 104 can be flushed of air prior to injecting medication, etc., through the second, inner needle 114. Thus it is advantageous if the plug 122 is sufficiently firmly lodged in the first, outer needle 108 so that a small pressure differential will not eject it. As described above, the internal surfaces of the first, outer needle 104 remain sterile until the plug 122 is either pierced or pushed aside by the deployment of the second needle 114.

It should be appreciated that a large variety of structures may be suitable for use as a gripping tool for holding the first, outer needle 108 during deployment. The housing 121 could be replaced by a handle or other holding mechanism if desired. Many means of making a fluid-tight communication between the second, inner needle 114 and the interior of the syringe body 123 are possible, including a Luer lock attachment or any other suitable mechanism for connecting the syringe to the needle.

Although the two needles 108 and 114 may form a substantially single, linear needle, the stylet or second, inner needle 114 need not be rigid, because the tissue pierced by the inner needle, if any, is typically softer and thinner than the tissue pierced by the first, outer needle 108. This allows the first, outer needle 108 to be bent or curved, so that when the outer needle 108 is advanced into a tissue, such as the conjunctiva, a linear puncture wound results. By providing a hypodermic needle system with bent or angled needles, several known complications with intravitreal injections may be minimized or eliminated, such as incision gaping, vitreous prolapse, vitreous bulge, and/or vitreous wick.

Attaching the syringe body 123 (or other medicament container) to the housing 121 or other gripping member prior to using the needle 108 to penetrate an exterior tissue surface may increase the ease of use of the hypodermic needle system. For example, it may be more difficult for medical personnel to advance the second, inner needle 114 with the syringe body attached into the first, outer needle 108, while the first, outer needle is in a patient.

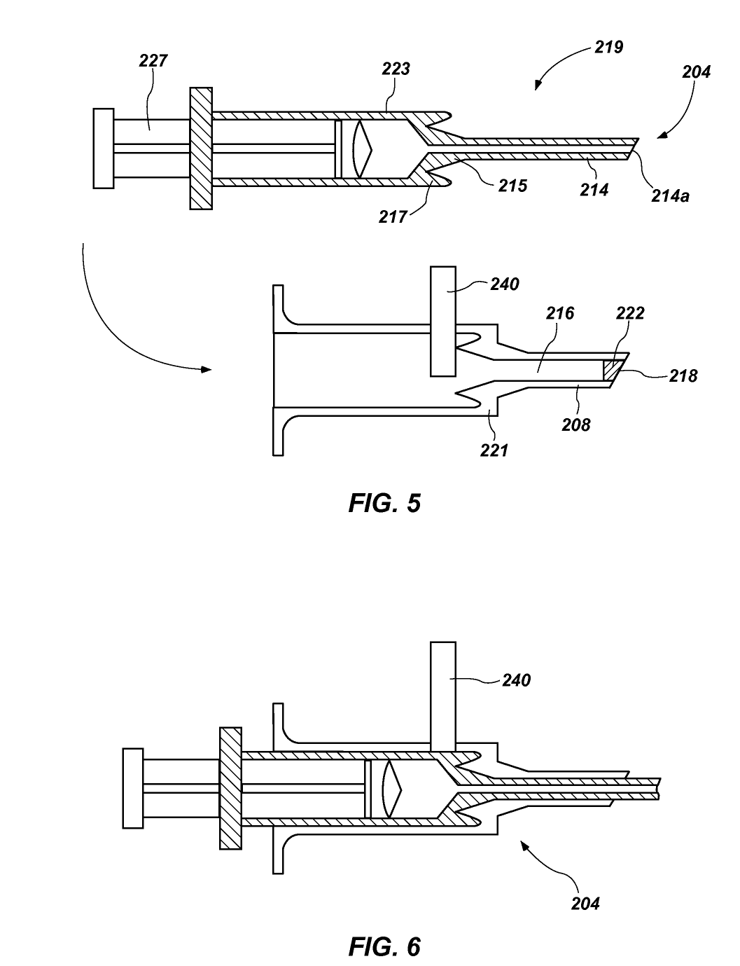

Turning now to FIG. 5, there is illustrated a side, exploded view of a two part hypodermic needle system, generally indicated at 204. One part includes a conventional syringe, generally indicated 219. The syringe 219 includes a syringe body 223, a plunger 227 which is moveable within the syringe body, and a needle 214 which is attached to the syringe body by a base 215 and a Luer lock 217 on the syringe body.

The other part of the syringe system 204 includes a housing or sheath 221 having a void configured to receive the syringe, and a needle 208 sized to receive the needle 214 of the syringe 219. The needle 208 is occluded at its distal end 218 by a plug 222. The length of the needle 208 is sufficiently shorter than the length of needle 214 that when the needle 214 is advanced in the needle 208, the needle 214 can push the plug 222 out of the distal end of the needle 208 or penetrate it centrally.

Also shown in FIG. 5 is a spacer member in the form of a stop 240 which selectively limits the advancement of the syringe 219 (typically by engaging the end of the Luer lock 217.) When the stop 240 is in a first, closed position, the stop prevents the advancement of the syringe so that the distal end 214a of the needle 214 cannot push the plug 222 out of the distal end 218 of the needle 208. Thus, medical personnel can advance the outer needle 208 by pushing on the syringe 219 without displacing the plug 222.

When the stop 240 is moved into a second, open position, as shown in FIG. 6, the syringe 219 can advance so that the distal end 214a of the needle 214 pushes the plug 222 out of the bore 216 in the needle 208 or penetrates it to thereby create a flow path past the plug. Thus, when the syringe 219 is loaded in the housing 221, the needle 208 is the first, outer needle, and the needle 214 is the second, inner needle. Having the stop 240 in the first, closed position prevents accidental opening of the distal end of the first, outer needle. For example, without the stop a technician could accidentally open the first, outer needle by pushing in the syringe too far. Because the end of syringes are small, he or she might not notice that the plug 222 has been pushed out, thereby losing the infection protection of the present invention. In contrast, maintaining the stop in the first, closed position until the first, outer needle 208 has been advanced through the skin, the technician, physician, etc., can be assured that the plug 222 is in place and preventing contamination of the bore 216. While the stop 240 is shown in the form of a slider, it will be appreciated that variety of other structures such as button, clamp or other restricting device could be used as well.

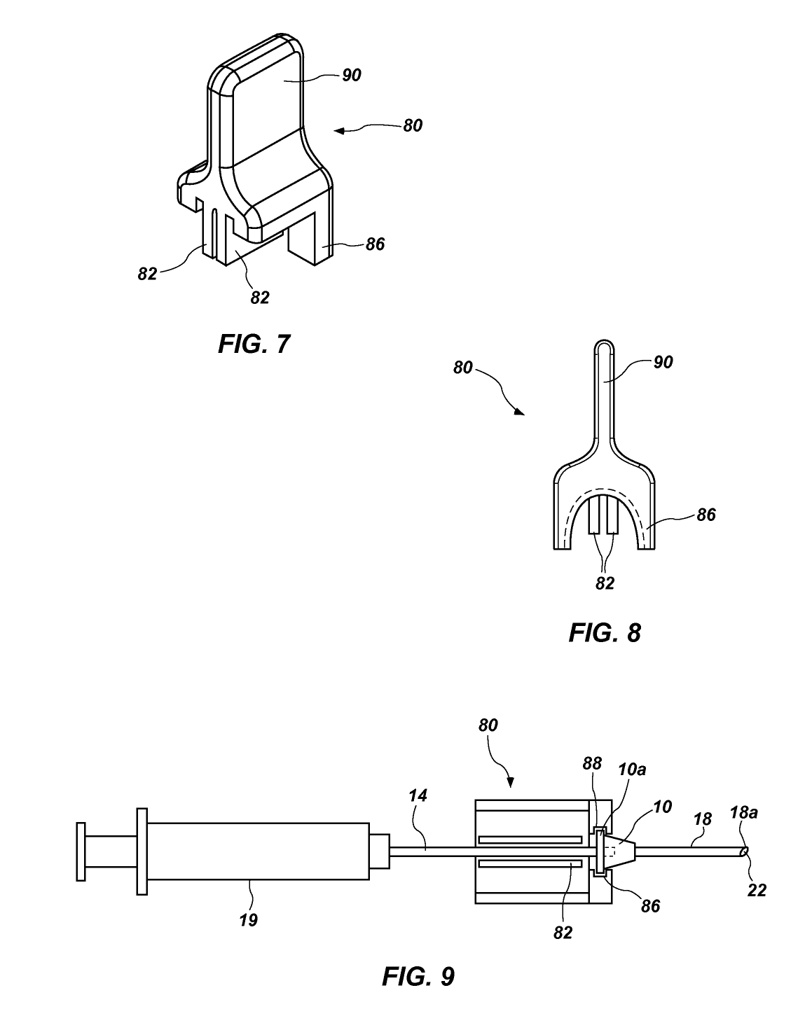

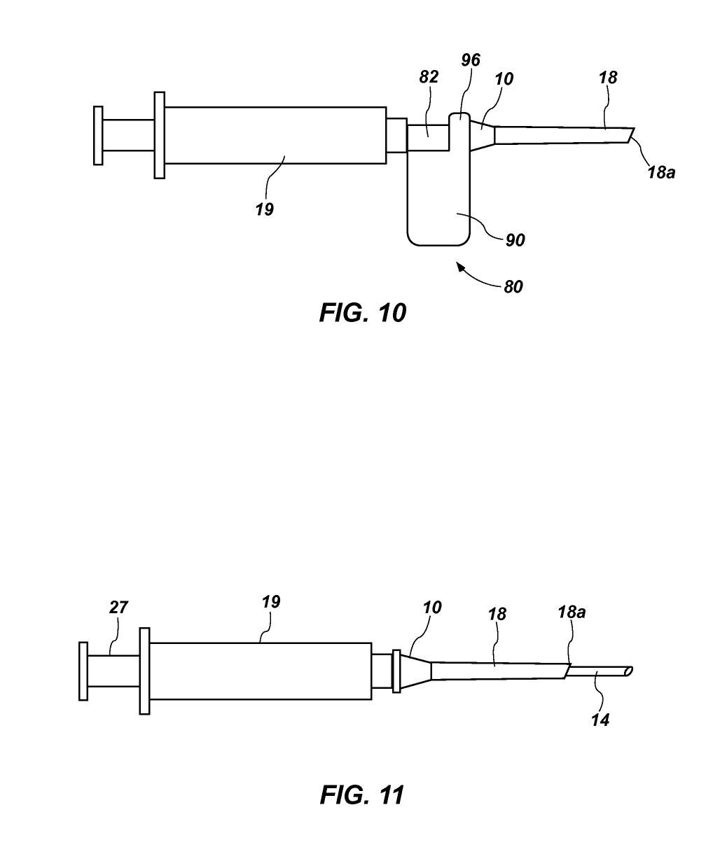

Turning now to FIGS. 7 and 8, there is shown, respectively, a bottom perspective a view and a bottom end view of a spacer member in the form of a spacer clip, indicated generally at 80, which may be used with a hypodermic needle system, such as system 4 above. The spacer clip 80 is adapted to hold a first, outer needle (18 (sideview FIG. 9 and top view FIG. 10) at a desired spacing from a second, inner needle (14, FIGS. 9 and 10). This embodiment of the spacer clip 80 may include, for example, one or more support arms 82, a holding portion 86 for receiving the base 10 (FIG. 1) attached to the first, outer needle, and a gripping portion 90 to facilitated attachment and removal of the spacing clip. The one or more support arms 82 are adapted to fit around the shaft of an internal needle (not shown), while the holding portion 86 has a channel 88 which receives a portion, such as the annular shoulder 10a (FIGS. 1 and 9) of the base 10 attached the first, outer needle.

The width of the spacer clip 90 holds the distal end of the second, inner needle 14 at a desired distance away from the plug 22 at the distal end 18a of the first, outer needle 18. When the releasing member 90 is manipulated or removed, the needle gripping prongs 82 disengage the inner needle 14 and end of the syringe body, allowing a user to advance the inner needle through the bore of the outer needle 18.

FIG. 9 shows a top view the second, inner needle 14 which forms part of the syringe 19 being advanced between the arms 82 of the spacer clip 90. The spacer clip 90 limits the extent to which the second, inner needle 14 can advance in the first, outer needle 18, which is held to the spacer clip 80 by the base 10 being held in a groove 88 in the attachment portion 86. FIG. 10 shows the syringe 19 fully advanced to the limit allowed by the spacer clip 90. In use the needle 18 would be advanced through the skin to the desired location and then the spacer clip 19 removed so that the second, inner needle 14 can be advanced and push the plug 22 out of the distal end 18a of the first, outer needle 18.

FIG. 11 shows a top view of the syringe 19, outer needle 18 and the housing 10, with the inner needle 14 fully advanced out the distal 18a of the outer needle. Thus, the inner needle 14 has either displaced or punctured through the plug 22. Drawing back the plunger 27 of the syringe 19, draws blood through the inner needle 14, thereby avoiding any contamination.

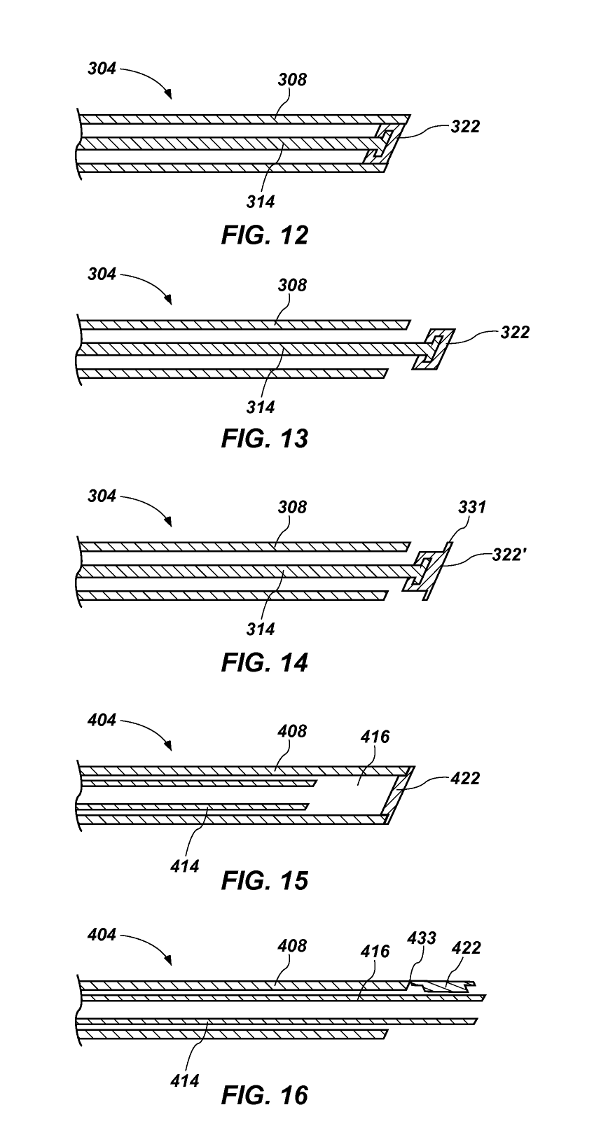

Turning now to FIGS. 12 and 13, there are shown an alternate embodiment of a two part hypodermic needle system, generally indicated at 304. Rather than using a plug which is ejected into the patient, the plug 322 is formed about the distal end of a stylet 314 disposed in the first, outer needle 308 The plug 322 may be formed from a sealing material, such as PLGA which can dissolve quickly in the body. The sealing material protects the bore 316 of the needle 308 during insertion. Once the needle 308 is properly positioned, the stylet 314 is advanced to move the plug out of the bore 316 as shown in FIG. 13 so that the needle 308 can be used to inject or to withdraw fluids from the body. Because of the lack of an open bore and mechanical wiping of the exterior of the plug 322 as it passes through tissue, the plug scoops or accumulates fewer bacteria than traditional syringes. Thus, the risk of contamination, either of the patient or a blood sample, is reduced.

Turning now to FIG. 14, there is shown a cross-sectional view of an alternate plug 322′. The plug 332′ is similar to the plug 332 in FIGS. 12 and 13 except that the plug may have a generally annular collar 331 or extension which extends outwardly so to cover the face of the distal end of the needle 308 when in the first, closed position. In such a manner the plug prevents or substantially reduces the risk of bacteria collecting along the face of the needle or along any seam between the plug and the outer needle.

FIGS. 15 and 16 show yet another embodiment of a two part hypodermic needle system, generally indicated at 404. The system 404 may include a first, outer needle 408 and an insert, such as a second, inner needle 414 which is advanceable within the first, outer needle. The first, outer needle 408 includes a bore 416 and a flap or plug 422 which selectively closes off the bore. As shown in FIG. 15, the plug 442 is disposed in a closed position, wherein the plug 422 closes the bore. As the needle 408 is advanced through tissue, the outside of the plug 422 is mechanically wiped by the tissue of the skin. Once in place, the second, inner needle 414 or other insert may be advanced to move the plug 422 out of the way as shown in FIG. 16, allowing fluid to be injected or withdrawn through either the first, outer needle 408 or the second, inner needle 414. (If a solid insert is used, the first, outer needle would be used for injecting or withdrawing fluid).

Unlike the prior embodiments, the plug 422 is neither ejected into the patient nor cored by the inner needle 414. Rather, the plug is attached like a flap so that it is simply pushed out of the way. The attachment mechanism 433 may be configured to bias the plug 422 back into the first, closed position, or may simply hold the plug to the needle with which it is withdrawn from the patient.

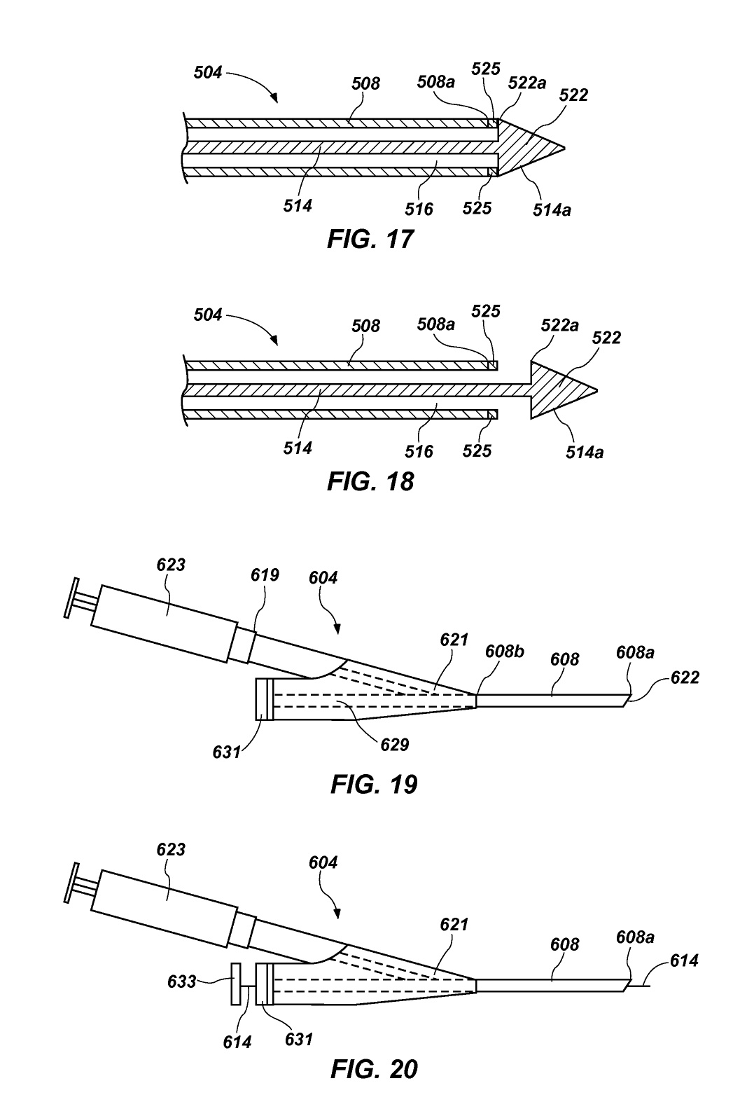

Turning now to FIGS. 17 and 18, there are shown a fragmented, side cross-sectional view of an alternate configuration of one aspect of the invention. The hypodermic needle system, generally indicated at 504, includes a first, outer needle 508 having a bore 516 therethrough, and an insert 514 in the form of a stylet disposed in the bore. The distal portion 514a of the stylet is enlarged and forms a plug 522 for selectively closing the bore 516. To facilitate a secure closing and minimize bacterial contamination of the bore 516, a seal 525 maybe placed between the distal end 508a of the first, outer needle 508 and the plug 522. The seal 525 may be generally annular and may be formed on or attached to either the distal end 508a of the first, outer needle 508 or to the proximal end 522a of the plug 522. The seal 525 may be made of a variety of materials. In one presently preferred embodiment, the seal 525 is made from a material which is resistant to adherence of bacteria and other microorganisms, such as PLGA.

Unlike the previous embodiments, the distal portion 522b of the plug 522 is tapered, beveled or otherwise comes to a point so as to facilitate puncturing in a manner similar to a conventional needle. The needle system 504 is used by advancing the plug 522 through the patient's skin and advancing it until the distal end 508a of the first, outer needle 508 is located at a depth desired by the person using the needle system. The insert or stylet 514 is then advanced sufficiently to form an opening between the plug 522 and the distal end of the first, outer needle 508. (A variety of gripping members or actuation devices may be used for selectively advancing the stylet 514 into the position shown in FIG. 18. For example, the first, outer needle 508 may be attached to a housing, and the stylet may be attached to a syringe which locks in place when advanced in the housing. The user may advance the first, outer needle 504 with the housing, and then advance the syringe when the first, outer needle 504 is at the desired depth).

Fluid in an attached syringe (not shown) may be injected through the opening between the first, outer needle 508 and the plug 522, or the plunger in an attached syringe may be pulled back to draw in a sample of a body fluid between the needle and the plug. Because of the pointed shape of the plug 522 and the seal 525, the risk of contamination in the bore 516 of the first, outer needle 508 is reduced. The skin through which the needle system 504 is penetrating will mechanically wipe the exterior of the plug 522, the seal 525 and the first, outer needle. Thus, the risk of bacteria or other micro-organisms getting inside of the bore 516 is significantly reduced.

FIGS. 19 and 20 show yet another embodiment of a hypodermic needle system, generally indicated at 604. The system includes a first, outer needle 608, which is attached to a housing 621 or other base structure on a proximal end 608b, and may have a plug 622 or membrane covering at the opposing distal end 608a The housing 621 may include a port 619 which is configured to engage a syringe 623 or other fluid storage device. The plug or membrane can be made from a material which resists adherence of bacteria and other micro-organisms, such as PLGA.

The housing 621 may also include a channel 629 through which a stylet or other insert 614 may advance so as to puncture or displace the plug 622 or membrane at the distal end of the needle. The distal end of the channel 629 may be covered with a seal, such as septum 631. As shown in FIG. 20, the stylet or insert 614 can pierce the septum 631 and be advanced until the distal end of the stylet punctures the plug 522 or membrane. Depending on the structure of the stylet or insert 614, the insert can remain in place, can be partially withdrawn, or may be completely withdrawn from the housing 621. It will be appreciated that the seal or septum 631 may be formed from self-healing rubber or other materials which will prevent any body fluids or injectable fluid from leaking through the septum. The stylet or insert 614 can be advanced or controlled by a gripping member such as the handle 633 shown in FIG. 20.

Because the distal end 608a of the first, outer needle 608 is covered by a material forming the plug 622, the bore of the needle (not shown) is not readily contaminated or filled with epidermal tissue. As the needle penetrates the skin, the first, outer needle 608 and the plug 622 are mechanically wiped by the tissues they pass through. Additionally the material of the plug may further reduce adherence of bacteria and the like. Once the first, outer needle 608 is in place, the stylet is advanced until it punctures the plug 622 or membrane and allows the syringe 623 to either inject or withdraw fluid. If the stylet is partially hollow or shaped, it may be able to remain in place during injection or draw of fluid. Otherwise, it can be partially or fully withdrawn once the plug 622 or membrane has been punctured.

While the various aspects of the invention are discussed with respect to individual embodiments, it will be appreciated that various aspects of one embodiment may be used with other embodiments and have been omitted for brevity.

While materials resistant to the adherence of bacteria and other micro-organisms are desirable to reduce contamination in either direction, materials such as PLGA are also beneficial in that they can be used to deliver medications. Thus, for example, a medication could be formed into the material which is used to form the plug. When the plug is ejected from the first, outer needle, not only is the first, outer needle or the second inner needle (when used as the insert) available to draw blood or inject medicine, the plug can also be a carrier of a medicine. This is particularly beneficial for time release medicines which can be contained within a material such as PLGA.

It will be appreciated that the present disclosure includes multiple inventions and aspects thereof, each of which may be independently patentable. For example, one aspect of the present disclosure includes a hypodermic needle system which may have a first needle having a bore and a distal end, the bore opening at the distal end; and a plug disposed in the bore, the plug being ejectable from the distal end, the plug being made from a bio-dissolvable material. The hypodermic needle system may further include: the plug having an outer face disposed outside of the first needle, and herein the outer face is convex; a stylet disposed in the bore of the first needle the stylet being advanceable to remove the plug from the bore or penetrate the plug; the style being a second needle having a bore therethrough; the first needle having a beveled edge and the plug comprises a beveled face and wherein the plug is disposed within the bore such that the beveled edge of said needle and the beveled face of said plug are substantially aligned, and wherein the beveled edge of the first needle and the beveled face of the plug substantially comprise a substantially planar surface; the second needle being adapted to advance independently of the first needle; a film disposed on at least one surface of the first needle; the film is being made of a substance selected from the group consisting of poly methyl methacrylate, cyanoacrylate, and polylactic-co-glycolic acid; the plug including polylactic-co-glycolic acid; an anti-infective agent comprised of at least one antiseptic; and/or the tip of the first needle is curved, and wherein the second needle is substantially flexible, and combinations thereof.

The disclosure also teaches a hypodermic needle system which may include: a first, outer needle having a bore therein; an insert disposed within the bore; and a plug for selectively sealing the bore of the first, outer needle. The system may further include: the plug being disposed inside the needle; the plug including a material which is resistant to the adherence of bacteria and other micro-organisms; the material of the plug being PLGA; the bore having a diameter and the plug having a diameter which is larger than the diameter of the bore and the plug remaining outside of the first, outer needle and/or the insert being a needle which is attached to a syringe body and a plunger, and combinations thereof.

The present disclosure also teaches a hypodermic needle system may include a first, outer needle having a bore therethrough and a plug for selectively closing the bore, and wherein the plug comprises bioabsorable matrix material have a therapeutic medication therein. The hypodermic needle system may further include; the therapeutic medication being a time-release medication; an insert disposed in the first, outer needle, the insert being movable within the first, outer needle to dislodge the plug from the first, outer needle; the insert being a needle having a hollow bore therethrough; and/or the hollow needle being attached to a syringe, and combinations thereof.

The present disclosure also teaches a hypodermic needle system which may include: a first needle having a bore and a distal end, the bore opening at the distal end; a stylet disposed in the bore; and a plug disposed in the bore and attached to the stylet. The hypodermic needle system may also include: the plug having an outer face disposed outside of the first needle, and the outer face being convex; a film attached to the plug; a film having a substance selected from the group consisting of poly methyl methacrylate, cyanoacrylate, and polylactic-co-glycolic acid; the plug being formed integrally with the stylet; the first needle having a beveled edge and the plug having a beveled face and the plug being disposed within the bore such that the beveled edge of said needle and the beveled face of said plug are substantially aligned, and the beveled edge of the first needle and the beveled face of the plug substantially having a planar surface; the stylet being adapted to advance independently of the first needle; the plug having a collar; an anti-infective agent comprised of at least one antiseptic disposed on the plug; a tip of the first needle being curved, and wherein the stylet is substantially flexible; and/or a housing attached to the first needle, the housing being adapted to receive a syringe, and combinations thereof.

The present disclosure also teaches a hypodermic needle system which may include: a first, outer needle having a bore therein; an insert disposed within the bore; and a plug for selectively sealing the bore of the first, outer needle, the plug further comprising a cutting edge. The system may also include the plug being formed on the insert such that movement of the insert moves the plug; the plug being disposed inside the needle; the plug having a material which is resistant to the adherence of bacteria and other micro-organisms; a film disposed over the plug; the bore having a diameter and the plug having a diameter which is larger than the diameter of the bore and wherein a portion of the plug remains outside of the first, outer needle; the plug having an convex outer surface faced away from the bore; a seal disposed between the first, outer needle and the plug; and/or the seal including an annular ring of PLGA.

The present disclosure also teaches a hypodermic needle system which may include: a first needle having a base, a bore and a distal end, the bore opening at the distal end; a plug disposed in the bore, the plug being ejectable from the distal end; an insert advanceable in the bore of the first needle; and a spacer clip disposable to limit advancement of the insert in the first needle to prevent the insert from ejecting the plug. The hypodermic needle may further include: the plug having an outer face disposed outside of the first needle, and the outer face being convex; the stylet being disposed in the bore of the first needle, the stylet being advanceable to remove the plug from the bore or to penetrate the plug; the stylet having a second needle having a bore therethrough; the first needle having a beveled edge and the plug having a beveled face and the plug being disposed within the bore such that the beveled edge of said needle and the beveled face of said plug are substantially aligned, and the beveled edge of the first needle and the beveled face of the plug having a substantially planar surface; the second needle being adapted to advance independently of the first needle; a syringe body attached to the insert and the spacer clip being configured to engage the syringe to limit forward movement of the insert; the spacer clip being configured to receive and hold the base of the first needle; the base having an annular shoulder and the spacer clip has a recess for receiving the annular shoulder to hold the base; an anti-infective agent comprised of at least one antiseptic disposed on or in the plug; a sleeve attached to the first needle, the sleeve being adapted to receive a syringe; the spacer clip being disposed along one of the sleeve or the syringe, the spacer clip being disposed to selectively limit advancement of the syringe within the sleeve; the spacer clip further comprising a gripping member adapted for engaging a syringe, a holding portion for receiving the first needle, and a releasing member for selectively limiting the positions of the first needle and the stylet; and/or the plug further having at least two layers, and combinations thereof.

The present disclosure also teaches a hypodermic needle system which may include: a first needle having a bore therein; an insert disposed within the bore; a spacer clip for selectively limiting the movement of the insert; and a plug for selectively sealing the bore of the first needle. The hypodermic needle system may further include: the plug being formed on the insert such that movement of the insert moves the plug; the plug being disposed inside the first needle; the plug having a material which is resistant to the adherence of bacteria and other micro-organisms; the plug including PLGA; the plug being is formed integrally with the insert; and/or a seal disposed between the first needle and the plug, and combinations thereof.

The present disclosure also teaches a spacer clip which may include: a gripping member adapted to receive a stylet; a holding portion adapted to receive a first needle; and a releasing member for selectively limiting the positions of the first needle and the stylet. The spacer clip may also include a connection mechanism adapted to connect to a syringe.

The present disclosure also teaches a method for reducing the risk of infection when injecting a human or animal, the method may include: selecting a first needle having a tip and a hollow bore and having an insert disposed within the hollow bore, and further having a plug disposed within the hollow bore so as to substantially prevent a contaminant from entering the hollow bore; advancing the needle into a tissue; advancing the insert so as to interrupt placement of the plug and thereby open a passage into the first needle; and passing a fluid through the first, outer needle. The method may also include: the step of applying an anti-infective agent to at least one surface of the first needle prior to advancing the first needle into a tissue to substantially eliminate any space where micro-organisms might be trapped; the tip of the first needle being bent so that advancing the needle into a tissue results in a substantially linear puncture wound; and/or the insert being a second needle and wherein the method comprises advancing the second needle in the first needle to eject the plug from the hollow bore of the first needle.

The disclosure also teaches a method for reducing the risk of infection when injecting a human or animal, the method may include: selecting a first needle having a tip and a hollow bore and having a second needle disposed within the hollow bore, and further having a plug disposed within the hollow bore so as to substantially prevent a contaminant from entering the hollow bore; advancing the needle into a tissue; advancing the second needle so as to interrupt placement of the plug and thereby open a passage into the hollow bore of the first, outer needle; and passing a fluid through one of the first, outer needle and the second, inner needle. The method may further include: the step of applying an anti-infective agent to at least one surface of the first needle prior to advancing the first needle into a tissue to substantially eliminate any space where micro-organisms might be trapped; the tip of the first needle being bent and the method includes advancing the needle into a tissue to form a substantially linear puncture wound; and/or the step of advancing the second needle so as to interrupt placement of the plug further comprises displacing or penetrating the plug with the second needle, and combinations thereof.

Likewise a method for forming a hypodermic needle system is taught which may include selecting a needle having a hollow bore disposed therein, the bore having at outlet and filling the outlet of the bore with a bio-dissolvable material to occlude the outlet. The method may further include: the bio-dissolvable material being flowable and curing the biocompatible solution in order to form a plug; and/or disposing a second needle inside the bore of the first needle, the second needle being of sufficient length that the needle can be advanced to push the plug out of the bore, and combinations thereof.

The disclosure also teaches a method for reducing the risk of infection when injecting a human or animal, the method may include: selecting a sleeve having: a spacer slider, a first needle having a tip and a hollow bore, and a plug disposed within the bore so as to substantially prevent a contaminant from entering the hollow bore; selecting a syringe having a second needle; inserting the second needle of the syringe into the first needle; advancing the first needle into a tissue; manipulating the spacer slider; advancing the second needle so as to interrupt placement of the plug and thereby open a passage into the first, outer needle; and passing a fluid through one of the first, outer needle and the second, inner needle. The method may further include: the step of applying an anti-infective agent to at least one surface of the first needle prior to advancing the first needle into a tissue to substantially eliminate any space where micro-organisms might be trapped; and/or the step of advancing the second needle so as to interrupt placement of the plug further comprises displacing or penetrating the plug with the second needle.

A method for reducing the risk of infection when injecting a human or animal, the method may also include one or more steps of: selecting needle hub having a port, a first needle having a tip and a hollow bore, and having a second needle disposed within the hollow bore, and further having a plug disposed within the hollow bore so as to substantially prevent a contaminant from entering the hollow bore; affixing a syringe to the port; advancing the first needle into a tissue; advancing the second needle so as to interrupt placement of the plug and thereby open a passage through the first, outer needle; and passing a fluid through one of the first, outer needle and the second, inner needle. The method may also include: applying an anti-infective agent to at least one surface of the first needle prior to advancing the first needle into a tissue; the first needle being bent so that advancing the needle into a tissue results in a substantially linear puncture wound; and/or advancing the second needle so as to interrupt placement of the plug further comprises displacing or penetrating the plug with the second needle, and combinations thereof.

The disclosure also teaches a hypodermic needle housing system which may include a needle hub with a port configured for attachment to a medicament container; a first needle having a bore and a distal end, the bore opening at the distal end; a plug disposed in the bore, the plug being moveable from the distal end; and a trigger mechanism configured to move the plug from the distal end. The housing system may also include: the plug having an outer face disposed outside of the first needle, and wherein the outer face is convex; a stylet disposed in the bore of the first needle, the stylet being advanceable to remove the plug from the bore or penetrate the bore; the stylet having a second needle having a bore therethrough; the first needle having a beveled edge and the plug comprises a beveled face and wherein the plug is disposed within the bore such that the beveled edge of said needle and the beveled face of said plug are substantially aligned, and wherein the beveled edge of the first needle and the beveled face of the plug substantially comprise a planar surface; the second needle being adapted to advance independently of the first needle; a film disposed on at least one surface of the first needle; the film being comprised of a substance selected from the group consisting of poly methyl methacrylate, cyanoacrylate, and polylactic-co-glycolic acid; the plug being comprised of polylactic-co-glycolic acid; an anti-infective agent comprised of at least one of the anti-infective agents selected from the group consisting of an antiseptic; the first needle being curved, and the second needle being substantially flexible; and/or at least one port of the needle hub is adapted to receive a syringe, and combinations thereof.

The disclosure also teaches a hypodermic needle system which may include: a needle hub having one or more ports; a first, outer needle having a bore therein; an insert disposed within the bore, the insert being longer than the first, outer needle; and a plug for selectively sealing the bore of the first, outer needle. The system may further include: a trigger mechanism for selectively advancing the insert independently of the first, outer needle; the plug comprises a material which is resistant to the adherence of bacteria and other micro-organisms; the material of the plug being PLGA; the plug being formed integrally with the insert; the bore having a diameter, the plug having a diameter which is larger than the diameter of the bore and the plug remaining outside of the first, outer needle; a seal disposed between the first, outer needle and the plug; a film disposed over the plug; the plug having a material which is resistant to the adherence of bacteria coated on the stylet; and/or the trigger mechanism being selected from the group consisting of: a button, squeeze lever, ratchet, or twistable extender.

The disclosure also teaches a hypodermic needle system which may include a sleeve having a first needle having a bore and a distal end, the bore having an opening at the distal end; a plug disposed in the opening of the bore, the plug being ejectable from the distal end; a connector for engaging a syringe; and a spacer slider for selectively limiting the position of the syringe in the sleeve. The system may also include; the plug having an outer face disposed outside of the first needle, and herein the outer face is convex; a stylet disposed in the bore of the first needle, the stylet being advanceable to remove the plug from the bore; the stylet having a second needle having a bore therethrough, and further having a lock for attaching the stylet to the syringe; the first needle comprises a beveled edge and the plug comprises a beveled face and wherein the plug is disposed within the bore such that the beveled edge of said needle and the beveled face of said plug are substantially aligned, and wherein the beveled edge of the first needle and the beveled face of the plug substantially comprise a planar surface; the second needle being adapted to advance independently of the first needle; the spacer slider being adapted to selectively engage the syringe; a film disposed on at least one surface of the first needle; a film comprised of a substance selected from the group consisting of poly methyl methacrylate, cyanoacrylate, and polylactic-co-glycolic acid; the plug having a time-release medication; and/or the first needle being curved, and the second needle being substantially flexible, and combinations thereof.

The present disclosure also teaches a hypodermic needle system which may include a sleeve having a spacer slider, the spacer slider being adapted to engage a syringe; a first needle having a bore therein, the first needle being attached to the sleeve; an insert disposed within the bore; and a plug for selectively sealing the bore of the first needle. The system may further include: the first needle and the insert forming a substantially air-tight seal; the first needle having a bore diameter, and the insert having an external diameter, the internal diameter being larger than the external diameter so as not to form an air-tight seal; the plug having a material which is resistant to the adherence of bacteria and other micro-organisms; the material being PLGA; wherein the insert is blunt; and/or a seal disposed between the first needle and the plug, and combinations thereof. Thus there is disclosed a two-needle injection system and methods of using the same. It will be appreciated that numerous modifications may be made without departing from the scope and spirit of this disclosure. The appended claims are intended to cover such modifications.

What is claimed is:

1. A hypodermic needle housing system comprising:
a needle hub with a port configured for attachment to a medicament container;
a first needle having a bore and a distal end, the bore opening at the distal end;
a plug disposed in the bore, the plug being moveable from the distal end; and
a trigger mechanism configured to move the plug from the distal end.

2. The hypodermic needle housing system of claim 1, wherein the plug has an outer face disposed outside of the first needle, and wherein the outer face is convex.

3. The hypodermic needle housing system of claim 1, further comprising a stylet disposed in the bore of the first needle, the stylet being advanceable to remove the plug from the bore or penetrate the bore.

4. The hypodermic needle housing system of claim 3, wherein the style comprises a second needle having a bore therethrough.

5. The hypodermic needle housing system according to claim 4, wherein the first needle comprises a beveled edge and the plug comprises a beveled face and wherein the plug is disposed within the bore such that the beveled edge of said needle and the beveled face of said plug are substantially aligned, and wherein the beveled edge of the first needle and the beveled face of the plug substantially comprise a planar surface.

6. The hypodermic needle housing system according to claim 4, wherein the second needle is adapted to advance independently of the first needle.

7. The hypodermic needle housing system of claim 1, further comprising a film disposed on at least one surface of the first needle.

8. The hypodermic needle housing system of claim 7, wherein the film is comprised of a substance selected from the group consisting of poly methyl methacrylate, cyanoacrylate, and polylactic-co-glycolic acid.

9. The hypodermic needle housing system of claim 1, wherein the plug is comprised of polylactic-co-glycolic acid.

10. The hypodermic needle housing system of claim 1, further comprising an anti-infective agent comprised of at least one of the anti-infective agents selected from the group consisting of an antiseptic.

11. The hypodermic needle housing system of claim 4, wherein the first needle is curved, and wherein the second needle is substantially flexible.

12. The hypodermic needle housing system of claim 1, wherein at least one port of the needle hub is adapted to receive a syringe.

13. A hypodermic needle system comprising:
a needle hub having one or more ports;
a first, outer needle having a bore therein;
an insert disposed within the bore, the insert being longer than the first, outer needle; and
a plug for selectively sealing the bore of the first, outer needle.

14. The hypodermic needle system of claim 13, further comprising a trigger mechanism for selectively advancing the insert independently of the first, outer needle.

15. The hypodermic needle system of claim 13, wherein the plug comprises a material which is resistant to the adherence of bacteria and other micro-organisms.

16. The hypodermic needle system of claim 13, wherein the material is PLGA.

17. The hypodermic needle system of claim 13, wherein the plug is formed integrally with the insert.

18. The hypodermic needle system of claim 17, wherein the bore has a diameter and wherein the plug has a diameter which is larger than the diameter of the bore and wherein the plug remains outside of the first, outer needle.

19. The hypodermic needle system of claim 18, further comprising a seal disposed between the first, outer needle and the plug.

20. The hypodermic needle system of claim 13, further comprising a film disposed over the plug.

21. The hypodermic needle system of claim 13, wherein the plug comprises a material which is resistant to the adherence of bacteria coated on the stylet.

22. The hypodermic needle system of claim 13, wherein the trigger mechanism is selected from the group consisting of: a button, squeeze lever, ratchet, or twistable extender.
